(19) United States
(12) Patent Application Publication
(10) Pub. No.: US 2024/0315933 A1
(43) Pub. Date: Sep. 26, 2024

(54) ALKALINE EARTH METAL CARBONATES FOR COSMIC COMPOSTIONS

(71) Applicant: IMERTECH SAS, Paris (FR)

(72) Inventors: Anaëlle ESPOSITO, Raphèle-les-Arles (FR); Elodie REMIA, Toulouse (FR); Alexandra JAKOB, Toulouse (FR); Cédric RABOTEAU, Fos Sur Mer (FR)

(21) Appl. No.: 18/261,192

(22) PCT Filed: Jan. 12, 2022
(86) PCT No.: PCT/EP2022/050568

§ 371 Date: Jul. 12, 2023

(30) Foreign Application Priority Data
Jan. 13, 2021 (EP) 21305029.7

(51) Int. Cl. A61K 8/19; A61K 8/02; A61Q 19/08

(52) U.S. Cl. CPC A61K 8/19; A61K 8/0245; A61Q 19/08; A61K 2800/622; A61K 2800/651

(57) ABSTRACT

An alkaline earth metal carbonate, for use in a cosmetic composition, has a BET specific surface area of at least about 60 m²/g. Particles of the alkaline earth metal carbonate are substantially spherical.

### TECHNICAL FIELD

The present disclosure concerns alkaline earth metal carbonates, cosmetic compositions comprising alkaline earth metal carbonates, uses of alkaline earth metal carbonates, and methods of reducing the appearance of skin imperfections.

### BACKGROUND

Cosmetic compositions such as makeup can be used to improve the appearance of skin, for example, by reducing the appearance of skin imperfections such as wrinkles. One way of reducing the appearance of skin imperfections, known as the soft focus effect, involves a scattered reflection of light incident on the skin. The soft focus effect of cosmetic compositions can blur unevenness, wrinkles and other skin imperfections, rendering them less visible. Cosmetic compositions typically include synthetic soft focus agents, such as poly(methyl methacrylate), nylon or synthetic silica to achieve the soft focus effect.

### SUMMARY OF THE INVENTION

According to a first aspect, there is provided an alkaline earth metal carbonate for use in a cosmetic composition, the alkaline earth metal carbonate having a BET specific surface area of at least about 60 m²/g, wherein particles of the alkaline earth metal carbonate are substantially spherical.

According to a second aspect, there is provided a cosmetic composition comprising the alkaline earth metal carbonate according to the first aspect and at least one other component.

According to a third aspect, there is provided a use of an alkaline earth metal carbonate having a BET specific surface area of at least about 60 m²/g in a cosmetic composition to improve the soft focus effect of the cosmetic composition, wherein particles of the alkaline earth metal carbonate are substantially spherical.

According to a fourth aspect, there is provided a method of reducing the appearance of skin imperfections, such as wrinkles, the method comprising applying (a) the alkaline earth metal carbonate according to the first aspect, or (b) the cosmetic composition according to the second aspect, to the skin.

According to a fifth aspect, there is provided an alkaline earth metal carbonate for use in a cosmetic composition, the alkaline earth metal carbonate having a BET specific surface area of at least about 60 m²/g and an agglomeration rate of at least about 85%, for example, at least about 90%, or at least about 92%, the agglomeration rate A being defined by

$$A = \frac{{D}_{50} @ {T}_{90}}{{D}_{50} @ {T}_{45}} \times 100 \% .$$

According to a sixth aspect, there is provided a cosmetic composition comprising the alkaline earth metal carbonate according to the fifth aspect and at least one other component.

According to a seventh aspect, there is provided a use of an alkaline earth metal carbonate having a BET specific surface area of at least about 60 m²/g in a cosmetic composition to improve the soft focus effect of the cosmetic composition, wherein the alkaline earth metal carbonate has an agglomeration rate of at least about 85%, for example, at least about 90%, or at least about 92%, the agglomeration rate A being defined by

$$A = \frac{{D}_{50} @ {T}_{90}}{{D}_{50} @ {T}_{45}} \times 100 \% .$$

According to an eighth aspect, there is provided a method of reducing the appearance of skin imperfections, such as wrinkles, the method comprising applying (a) the alkaline earth metal carbonate according to the fifth aspect, or (b) the cosmetic composition according to the sixth aspect, to the skin.

The skilled person will appreciate that, except where mutually exclusive, a feature described in relation to any one of the above aspects may be applied mutatis mutandis to any other aspect. Furthermore, except where mutually exclusive, any feature described herein may be applied to any aspect and/or combined with any other feature described herein.

In each of the aspects, the following parameters are preferably measured as follows.

The BET particle surface area may be measured according to the BET method, as in ISO 9277.

The D₅₀@T₄₅ and D₅₀@T₉₀ may be determined by a method known as “dispersion evaluation by laser method” (DELM) using laser granulometry using the Malvern Mastersizer 2000 (as supplied by Malvern instruments), as described in more detail hereinbelow.

### FIGURES

Embodiments will now be described by way of example only, with reference to the Figures, in which:

FIG. 1 is a Scanning Electron Microscope (SEM) image of particles of example PCC1;

FIG. 2 is an SEM image of particles of example PCC9;

FIG. 3 is an SEM image of particles of example PCC13;

FIG. 4 is an SEM image of particles of example PCC4;

FIG. 5 is an SEM image of particles of example PCC6;

FIG. 6 is an SEM image of particles of example PCC7;

FIG. 7 is a plot of BET surface area as a function of soft focus effect score for various PCCs;

FIG. 8 is a plot of D₅₀@T₉₀ as a function of soft focus effect score for various PCCs;

FIG. 9 is a plot of agglomeration rate as a function of soft focus effect score for various PCCs; and

FIG. 10 is the haze results of example PCC21.

### DETAILED DESCRIPTION OF THE INVENTION

It has surprisingly been found that alkaline earth metal carbonates having a high BET specific surface area (typically of at least about 60 m²/g) can improve the soft focus effect of cosmetic compositions.

### Alkaline Earth Metal Carbonates

The alkaline earth metal carbonate may be a carbonate of a single alkaline earth metal. Alternatively, the alkaline earth metal carbonate may comprise carbonates of two or more different alkaline earth metals. The alkaline earth metal carbonate (for example, each alkaline earth metal carbonate) may comprise carbonates of one or more of the following: beryllium, magnesium, calcium, strontium, barium, radium.

The alkaline earth metal carbonate may be calcium carbonate. The alkaline earth metal carbonate may be precipitated calcium carbonate (PCC). Precipitated calcium carbonate (PCC) may be made by any method known in the art.

TAPPI Monograph Series No 30, “Paper Coating Pigments”, pages 34-35, the contents of which are incorporated herein by reference, describes three main commercial processes for preparing precipitated calcium carbonate. In all three processes, limestone is first calcined to produce quicklime, and the quicklime is then slaked in water to yield calcium hydroxide or milk of lime. In the first process, the milk of lime is directly carbonated with carbon dioxide gas. This process has the advantage that little or no by-product is formed, and it is relatively easy to control the properties and purity of the calcium carbonate product. In the second process, the milk of lime is contacted with soda ash to produce, by double decomposition, a precipitate of calcium carbonate and a solution of sodium hydroxide. Typically, the sodium hydroxide is substantially completely separated from the calcium carbonate. In the third main commercial process, the milk of lime is first contacted with ammonium chloride to give a calcium chloride solution and ammonia gas. The calcium chloride solution is then contacted with soda ash to produce, by double decomposition, precipitated calcium carbonate and a solution of sodium chloride.

Alternatively, PCC may be made by reacting gypsum (calcium sulphate) with ammonium carbonate or ammonium bicarbonate.

Alternatively, PCC may be made by reacting calcium chloride with sodium carbonate or ammonium carbonate.

Alternatively, PCC may be obtained by carbonation of milk of lime in the presence of a crystallization controller selected from: polyacrylic acid, an aminopolycarboxylic acid (e.g. EDTA), citric acid, and salts thereof; aluminium sulfate; saccharose (e.g. sucrose);

and any mixtures thereof. For instance, PCC may be prepared by one or more of the methods described in WO03/004414, the contents of which are incorporated herein by reference, particularly page 2, line 11 to page 3, line 38; page 4, line 29 to page 5, line 6; and page 5, line 36 to page 6, line 28 as well as examples 4 and 5.

In the preparation process, the concentration of calcium hydroxide in the milk of lime can have a value of from about 0.3 wt. % to about 30.0 wt. %, for example, at least about 1.0 wt. %, or at least about 2.0 wt. %, or at least about 2.5 wt. %, or at least about 4.0 wt. %, or at least about 5.0 wt. %, or at least about 6.0 wt. %, or at least about 7.0 wt. %, or at least about 8.0 wt. %, based on the weight of the milk of lime. It is recommended that the concentration of calcium hydroxide in the milk of lime does not exceed about 25.0 wt. %, for example, about 20.0 wt. %, or about 16.0 wt. %, or about 10 wt. %, or about 9 wt. %. For instance, the concentration of calcium hydroxide in the milk of lime might be from about 0.3 wt. % to about 30.0 wt. %, for example, from about 1.0 wt. % to about 30.0 wt. %, or from about 2.0 wt. % to about 30.0 wt. %, or from about 2.5 wt. % to about 30.0 wt. %, or from about 4.0 wt. % to about 30.0 wt. %, or from about 5.0 wt. % to about 30.0 wt. %, or from about 6.0 wt. % to about 30.0 wt. %, or from about 7.0 wt. % to about 30.0 wt. %, or from about 8.0 wt. % to about 30.0 wt. %, or from about 0.3 wt. % to about 20.0 wt. %, or from about 1.0 wt. % to about 20.0 wt. %, or from about 2.0 wt. % to about 20.0 wt. %, or from about 2.5 wt. % to about 20.0 wt. %, or from about 4.0 wt. % to about 20.0 wt. %, or from about 5.0 wt. % to about 20.0 wt. %, or from about 6.0 wt. % to about 20.0 wt. %, or from about 7.0 wt. % to about 20.0 wt. %, or from about 8.0 wt. % to about 20.0 wt. %, or from about 0.3 wt. % to about 16.0 wt. %, or from about 1.0 wt. % to about 16.0 wt. %, or from about 2.0 wt. % to about 16.0 wt. %, or from about 2.5 wt. % to about 16.0 wt. %, or from about 4.0 wt. % to about 16.0 wt. %, or from about 5.0 wt. % to about 16.0 wt. %, or from about 6.0 wt. % to about 16.0 wt. %, or from about 7.0 wt. % to about 16.0 wt. %, or from about 8.0 wt. % to about 16.0 wt. %, or from about 0.3 wt. % to about 10.0 wt. %, or from about 1.0 wt. % to about 10.0 wt. %, or from about 2.0 wt. % to about 10.0 wt. %, or from about 2.5 wt. % to about 10.0 wt. %, or from about 4.0 wt. % to about 10.0 wt. %, or from about 5.0 wt. % to about 10.0 wt. %, or from about 6.0 wt. % to about 10.0 wt. %, or from about 7.0 wt. % to about 10.0 wt. %, or from about 8.0 wt. % to about 10.0 wt. %, or from about 0.3 wt. % to about 9.0 wt. %, or from about 1.0 wt. % to about 9.0 wt. %, or from about 2.0 wt. % to about 9.0 wt. %, or from about 2.5 wt. % to about 9.0 wt. %, or from about 4.0 wt. % to about 9.0 wt. %, or from about 5.0 wt. % to about 9.0 wt. %, or from about 6.0 wt. % to about 9.0 wt. %, or from about 7.0 wt. % to about 9.0 wt. %, or from about 8.0 wt. % to about 9.0 wt. %, or about 8.0 wt. %.

In said preparation process, the temperature may vary from about 0° C. to about 80° C., for example, from about 10° C. to about 60° C. Usually, the temperature at the beginning of carbonation is equal to or higher than about 10° C., for example, equal to or higher than about 12° C. The temperature at the beginning of carbonation is typically equal to or lower than about 30° C., for example, equal to or lower than about 25° C., or equal to or lower than about 20° C. The temperature at the beginning of carbonation might for instance be about 12° C., or about 15° C., or about 18° C. The temperature at the end of carbonation might be higher, for example, from about 10° C. to about 80° C., or from about 15° C. to about 65° C., or from about 35° C. to about 65° C.

In the preparation process, milk of lime is carbonated by reaction of the latter with carbon dioxide gas. Carbon dioxide gas having a concentration of carbon dioxide varying from about 3.0% to about 100% could be used with success. However, it is typical to use carbon dioxide gas for which the concentration is from about 10% to 60%, for example, from about 15% to about 40%, or from about 20% to about 30%, the carbon dioxide gas being diluted with air.

Some additives might also be further added during the carbonation step, such as isoascorbic acid, to increase brightness (for example, reduce yellowness) of the resulting calcium carbonate particles. Said preparation process typically leads to a precipitated calcium carbonate slurry comprising for instance about 3.0 wt. % to about 25.0 wt. % of PCC, based on the weight of the slurry.

Precipitated calcium carbonate particles might be filtered, for example through a planar filter, and dried, for instance in an oven, by spraying into a stream of hot air (spray drying), or by the action of radiation such as infrared radiation (epiradiator), typically in an oven or by the action of radiation such as infrared radiation. The resulting particles might then be further milled, for instance in a pin mill apparatus.

Such processes for making PCC typically result in very pure calcium carbonate crystals, which may be referred to as “elementary” or “primary” particles or crystallites. In this context, the terms “elementary particle”, “elementary crystallite”, “primary particle” or “primary crystallite” refer to a physically and chemically autonomous entity. The elementary or primary particles or crystallites may have a variety of different shapes and sizes, depending on the specific reaction process that is used. Mixtures of different morphologies may also be used.

The main polymorphs of PCC crystals are aragonite and calcite. Aragonite crystals are needle-shaped and may be randomly aggregated. Calcite crystals may be pseudo-spherical, cubic or scalenohedral in morphology. For example, calcite crystals may have rhombohedral morphology.

The alkaline earth metal carbonate, for example, calcium carbonate (e.g., PCC), typically has a BET specific surface area of at least about 60 m²/g. Such an alkaline earth metal carbonate, when used in a cosmetic composition, shows an improved soft focus effect compared to a similar alkaline earth metal carbonate having a BET specific surface area of less than about 60 m²/g. The alkaline earth metal carbonate may have a BET specific surface area of at least about 64 m²/g, for example, at least about 65 m²/g, or at least about 70 m²/g, or at least about 75 m²/g, or at least about 80 m²/g. The alkaline earth metal carbonate, for example, calcium carbonate (e.g., PCC), may have a BET specific surface area of no greater than about 200 m²/g, for example, no greater than about 150 m²/g, or no greater than about 140 m²/g, or no greater than about 120 m²/g, or no greater than about 100 m²/g. The alkaline earth metal carbonate, for example, calcium carbonate (e.g., PCC), may have a BET specific surface area of from about 60 m²/g to about 200 m²/g, for example, from about 64 m²/g to about 200 m²/g, or from about 65 m²/g to about 200 m²/g, or from about 70 m²/g to about 200 m²/g, or from about 75 m²/g to about 200 m²/g, or from about 80 m²/g to about 200 m²/g, or from about 60 m²/g to about 150 m²/g, or from about 64 m²/g to about 150 m²/g, or from about 65 m²/g to about 150 m²/g, or from about 70 m²/g to about 150 m²/g, or from about 75 m²/g to about 150 m²/g, or from about 80 m²/g to about 150 m²/g, or from about 60 m²/g to about 140 m²/g, or from about 64 m²/g to about 140 m²/g, or from about 65 m²/g to about 140 m²/g, or from about 70 m²/g to about 140 m²/g, or from about 75 m²/g to about 140 m²/g, or from about 80 m²/g to about 140 m²/g, or from about 60 m²/g to about 120 m²/g, or from about 64 m²/g to about 120 m²/g, or from about 65 m²/g to about 120 m²/g, or from about 70 m²/g to about 120 m²/g, or from about 75 m²/g to about 120 m²/g, or from about 80 m²/g to about 120 m²/g, or from about 60 m²/g to about 100 m²/g, or from about 64 m²/g to about 100 m²/g, or from about 65 m²/g to about 100 m²/g, or from about 70 m²/g to about 100 m²/g, or from about 75 m²/g to about 100 m²/g, or from about 80 m²/g to about 100 m²/g.

Particle size properties may be measured in a well-known manner by sedimentation of the particulate filler or material in a fully dispersed condition in an aqueous medium using a Sedigraph 5100 machine as supplied by Micromeritics Instruments Corporation, Norcross, Georgia, USA (web-site: www.micromeritics.com), referred to herein as a “Micromeritics Sedigraph 5100 unit”. Such a machine provides measurements and a plot of the cumulative percentage by weight of particles having a size, referred to in the art as the ‘equivalent spherical diameter’ (e.s.d), less than given e.s.d values. The mean particle size D₅₀ is the value determined in this way of the particle e.s.d at which there are 50% by weight of the particles which have an equivalent spherical diameter less than that D₅₀ value. The D₉₈. D₉₀ and the D₁₀ are the values determined in this way of the particle e.s.d. at which there are 98%, 90% and 10% respectively by weight of the particles which have an equivalent spherical diameter less than that D₉₈, D₉₀ or D₁₀ value. Particle size properties may also be measured by wet Malvern laser scattering (standard ISO 13320-1). In this technique, the size of particles in powders, suspensions and emulsions may be measured using the diffraction of a laser beam, based on the application of Mie theory. Such a machine, for example a Malvern Mastersizer S or a Malvern Mastersizer 2000 (as supplied by Malvern instruments) provides measurements and a plot of the cumulative percentage by volume of particles having a size, referred to in the art as the “equivalent spherical diameter” (e.s.d), less than given e.s.d values. The mean particle size D₅₀ is the value determined in this way of the particle e.s.d. at which there are 50% by volume of the particles which have an equivalent spherical diameter less than that D₅₀ value. For the avoidance of doubt, the measurement of particle size using laser light scattering is not an equivalent method to the sedimentation method referred to above.

The dispersion behaviour of a particulate material (such as an agglomerated particulate material) can be characterised by the parameters D₅₀@T₄₅ and/or D₅₀@T₉₀. D₅₀@T₄₅ and D₅₀@T₉₀ can be determined by a method known as “dispersion evaluation by laser method” (DELM) using laser granulometry, for example, using the Malvern Mastersizer 2000 (as supplied by Malvern instruments). The DELM method for alkaline earth metal carbonates is based on the laser granulometry method for precipitated silica described in U.S. Pat. No. 6,610,261 by Michelin. In the DELM method, the measurement cell of a wet Malvern laser scattering device (e.g. the Malvern Mastersizer 2000) is filled with a mixture of isopropanol and a small amount of alkaline earth metal carbonate. The obscuration is then measured and should be between 1 and 3% for the measurement to be reliable. The mixture is then subjected to mechanical stirring at about 1500 rpm for about 4 minutes and 30 seconds. The particle size distribution (PSD) is measured 45 times during this time. The 45^{th} PSD, or the nearest representative PSD, is recorded. A PSD is considered as representative when it does not comprise agglomerates above 500 μm which could be attributed to air bubbles. The D₅₀ value obtained from the recorded PSD after stirring the mixture is known as the D₅₀@T₄₅.

Following measurement of D₅₀@T₄₅, the stirred mixture is subjected to pulsed ultrasound for about 4 minutes and 30 seconds at a total intensity of about 1000 J to 1500 J. The PSD is again measured 45 times during this time. The 45^{th} PSD, or the nearest representative PSD, is recorded. A PSD is considered as representative when it does not comprise agglomerates above 500 μm which could be attributed to air bubbles. The D₅₀ value obtained from the recorded PSD after application of the pulsed ultrasound is known as the D₅₀@T₉₀. The pulsed ultrasound may be applied using a sonicator, for example, the Vibracell 75186 sonicator (available from Thermo Fisher Scientific, Inc., USA), operated at a power of 130 W and a frequency of 20 kHz, with a probe having a 3 mm stepped tip. The intensity of the pulsed ultrasound is selected by measuring a PCC reference sample and ensuring that the D₅₀@T₉₀ is +/−10% of what has been obtained previously.

The alkaline earth metal carbonate, for example, calcium carbonate (e.g., PCC), may have a D₅₀@T₉₀ no less than about 6 μm, for example, no less than about 7 μm, or no less than about 8 μm, or no less than about 9 μm, or no less than about 10 μm. The alkaline earth metal carbonate, for example, calcium carbonate (e.g., PCC), may have a D₅₀@T₉₀ no greater than about 20 μm, for example, no greater than about 19 μm, or no greater than about 18 μm, or no greater than about 17 μm, or no greater than about 16 μm, or no greater than about 15 μm. The alkaline earth metal carbonate, for example, calcium carbonate (e.g., PCC), may have a D₅₀@T₉₀ from about 6 μm to about 20 μm, for example, from about 7 μm to about 20 μm, or from about 8 μm to about 20 μm, or from about 9 μm to about 20 μm, or from about 10 μm to about 20 μm, or from about 6 μm to about 19 μm, or from about 7 μm to about 19 μm, or from about 8 μm to about 19 μm, or from about 9 μm to about 19 μm, or from about 10 μm to about 19 μm, or from about 6 μm to about 18 μm, or from about 7 μm to about 18 μm, or from about 8 μm to about 18 μm, or from about 9 μm to about 18 μm, or from about 10 μm to about 18 μm, or from about 6 μm to about 17 μm, or from about 7 μm to about 17 μm, or from about 8 μm to about 17 μm, or from about 9 μm to about 17 μm, or from about 10 μm to about 17 μm, or from about 6 μm to about 16 μm, or from about 7 μm to about 16 μm, or from about 8 μm to about 16 μm, or from about 9 μm to about 16 μm, or from about 10 μm to about 16 μm, or from about 6 μm to about 15 μm, or from about 7 μm to about 15 μm, or from about 8 μm to about 15 μm, or from about 9 μm to about 15 μm, or from about 10 μm to about 15 μm.

The alkaline earth metal carbonate, for example, calcium carbonate (e.g., PCC), may have a D₅₀@T₄₅ no less than about 5 μm, for example, no less than about 7 μm, or no less than about 10 μm. The alkaline earth metal carbonate, for example, calcium carbonate (e.g., PCC), may have a D₅₀@T₄₅ no greater than about 40 μm, for example, no greater than about 30 μm, or no greater than about 20 μm, or no greater than about 15 μm. The alkaline earth metal carbonate, for example, calcium carbonate (e.g., PCC), may have a D₅₀@T₄₅ from about 5 μm to about 40 μm, for example, from about 7 μm to about 40 μm, or from about 10 μm to about 40 μm, or from about 5 μm to about 30 μm, or from about 7 μm to about 30 μm, or from about 10 μm to about 30 μm, or from about 5 μm to about 20 μm, or from about 7 μm to about 20 μm, or from about 10 μm to about 20 μm, or from about 5 μm to about 15 μm, or from about 7 μm to about 15 μm, or from about 10 μm to about 15 μm.

The alkaline earth metal carbonate, for example, calcium carbonate (e.g., PCC), may have an agglomeration rate of at least about 85%, for example, at least about 90%, or at least about 92%, or at least about 95%, or at least about 99%, the agglomeration rate A being defined by

$$A = \frac{{D}_{50} @ {T}_{90}}{{D}_{50} @ {T}_{45}} \times 100 \% .$$

An alkaline earth metal carbonate having an agglomeration rate of at least about 85%, when used in a cosmetic composition, shows an improved soft focus effect compared to a similar alkaline earth metal carbonate having an agglomeration rate of less than about 85%.

The agglomeration rate of the alkaline earth metal carbonate, for example, calcium carbonate (e.g., PCC), may be no greater than about 102%, for example, no greater than about 100%, or no greater than about 99%, or no greater than about 95%. The agglomeration rate of the alkaline earth metal carbonate, for example, calcium carbonate (e.g., PCC), may be from about 85% to about 102%, for example, from about 90% to about 102%, or from about 92% to about 102%, or from about 95% to about 102%, or from about 99% to about 102%, or from about 85% to about 100%, or from about 90% to about 100%, or from about 92% to about 100%, or from about 95% to about 100%, or from about 99% to about 100%, or from about 85% to about 99%, or from about 90% to about 99%, or from about 92% to about 99%, or from about 85% to about 95%, or from about 90% to about 95%, or from about 92% to about 95%.

It may be that the alkaline earth metal carbonate, for example, calcium carbonate (e.g., PCC), has: a BET specific surface area from about 60 m²/g to about 200 m²/g, for example, from about 64 m²/g to about 200 m²/g, or from about 65 m²/g to about 200 m²/g, or from about 70 m²/g to about 200 m²/g, or from about 75 m²/g to about 200 m²/g, or from about 80 m²/g to about 200 m²/g, or from about 60 m²/g to about 150 m²/g, or from about 64 m²/g to about 150 m²/g, or from about 65 m²/g to about 150 m²/g, or from about 70 m²/g to about 150 m²/g, or from about 75 m²/g to about 150 m²/g, or from about 80 m²/g to about 150 m²/g, or from about 60 m²/g to about 140 m²/g, or from about 64 m²/g to about 140 m²/g, or from about 65 m²/g to about 140 m²/g, or from about 70 m²/g to about 140 m²/g, or from about 75 m²/g to about 140 m²/g, or from about 80 m²/g to about 140 m²/g, or from about 60 m²/g to about 120 m²/g, or from about 64 m²/g to about 120 m²/g, or from about 65 m²/g to about 120 m²/g, or from about 70 m²/g to about 120 m²/g, or from about 75 m²/g to about 120 m²/g, or from about 80 m²/g to about 120 m²/g, or from about 60 m²/g to about 100 m²/g, or from about 64 m²/g to about 100 m²/g, or from about 65 m²/g to about 100 m²/g, or from about 70 m²/g to about 100 m²/g, or from about 75 m²/g to about 100 m²/g, or from about 80 m²/g to about 100 m²/g; a D₅₀@T₉₀ from about 6 μm to about 20 μm, for example, from about 7 μm to about 20 μm, or from about 8 μm to about 20 μm, or from about 9 μm to about 20 μm, or from about 10 μm to about 20 μm, or from about 6 μm to about 19 μm, or from about 7 μm to about 19 μm, or from about 8 μm to about 19 μm, or from about 9 μm to about 19 μm, or from about 10 μm to about 19 μm, or from about 6 μm to about 18 μm, or from about 7 μm to about 18 μm, or from about 8 μm to about 18 μm, or from about 9 μm to about 18 μm, or from about 10 μm to about 18 μm, or from about 6 μm to about 17 μm, or from about 7 μm to about 17 μm, or from about 8 μm to about 17 μm, or from about 9 μm to about 17 μm, or from about 10 μm to about 17 μm, or from about 6 μm to about 16 μm, or from about 7 μm to about 16 μm, or from about 8 μm to about 16 μm, or from about 9 μm to about 16 μm, or from about 10 μm to about 16 μm, or from about 6 μm to about 15 μm, or from about 7 μm to about 15 μm, or from about 8 μm to about 15 μm, or from about 9 μm to about 15 μm, or from about 10 μm to about 15 μm; and an agglomeration rate, as defined hereinabove, from about 85% to about 102%, for example, from about 90% to about 102%, or from about 92% to about 102%, or from about 95% to about 102%, or from about 99% to about 102%, or from about 85% to about 100%, or from about 90% to about 100%, or from about 92% to about 100%, or from about 95% to about 100%, or from about 99% to about 100%, or from about 85% to about 99%, or from about 90% to about 99%, or from about 92% to about 99%, or from about 85% to about 95%, or from about 90% to about 95%, or from about 92% to about 95%.

It may be that the alkaline earth metal carbonate, for example, calcium carbonate (e.g., PCC), has: (a) a BET specific surface area from about 60 m²/g to about 200 m²/g, for example, from about 64 m²/g to about 140 m²/g; (b) a D₅₀@T₉₀ of at least about 6 μm, for example, from about 6 μm to about 20 μm; and/or (c) an agglomeration rate, as defined hereinabove, of at least about 85%, for example, at least about 90%, or at least about 92%.

It may be that the alkaline earth metal carbonate, for example, calcium carbonate (e.g., PCC), has: (a) a BET specific surface area from about 60 m²/g to about 100 m²/g, for example, from about 70 m²/g to about 80 m²/g; (b) a D₅₀@T₉₀ of at least about 10 μm, for example, from about 10 μm to about 20 μm, or from about 13 μm to about 17 μm; and/or (c) an agglomeration rate, as defined hereinabove, of least about 95%, for example, at least about 99%.

The alkaline earth metal carbonate, for example, calcium carbonate (e.g., PCC), typically comprises a plurality of particles. It may be that particles of the alkaline earth metal carbonate, for example, calcium carbonate (e.g., PCC), are substantially spherical (i.e. in shape). Such a substantially spherical alkaline earth metal carbonate (i.e. an alkaline earth metal carbonate having particles which are substantially spherical), when used in a cosmetic composition, shows an improved soft focus effect compared to a non-spherical similar alkaline earth metal carbonate (i.e. a similar alkaline earth metal carbonate having particles which are substantially non-spherical). For example, it may be that no less than about 50 wt. %, for example, no less than about 60 wt. %, or no less than about 70 wt. %, or no less than about 80 wt. %, or no less than about 90 wt. %, or no less than about 95 wt. %, or no less than about 99 wt. %, of the particles of the alkaline earth metal carbonate, for example, calcium carbonate (e.g., PCC), are substantially spherical. It may be that substantially all (i.e. about 100 wt. %) of the particles of the alkaline earth metal carbonate, for example, calcium carbonate (e.g., PCC), are substantially spherical, i.e. that the alkaline earth metal carbonate, for example, calcium carbonate (e.g., PCC), consists essentially of substantially spherical particles.

It will be appreciated that the substantially spherical particles are not necessarily entirely, or precisely, spherical. For example, the substantially spherical particles may include (e.g. be) pseudo-spherical particles.

Particle sphericity may be quantified in terms of particle circularity and/or elongation. The particles of the alkaline earth metal carbonate, for example, calcium carbonate (e.g., PCC), may have a circularity (e.g. a high sensitivity (HS) circularity) of no less than about 0.8 (e.g. no less than about 0.80), for example, no less than about 0.81, or no less than about 0.82, or no less than about 0.83, or no less than about 0.84, or no less than about 0.85, or no less than about 0.86, or no less than about 0.87, or no less than about 0.88, or no less than about 0.89, or no less than about 0.9 (e.g. no less than about 0.90), or no less than about 0.91, or no less than about 0.92, or no less than about 0.93, or no less than about 0.94, or no less than about 0.95. The particles of the alkaline earth metal carbonate, for example, calcium carbonate (e.g., PCC), may have an elongation of no greater than about 0.2 (e.g. no greater than about 0.20), for example, no greater than about 0.19, or no greater than about 0.18, or no greater than about 0.17, or no greater than about 0.16, or no greater than about 0.15, or no greater than about 0.14, or no greater than about 0.13, or no greater than about 0.12, or no greater than about 0.11, or no greater than about 0.1 (e.g. no greater than about 0.10), or no greater than about 0.09, or no greater than about 0.08, or no greater than about 0.07, or no greater than about 0.06, or no greater than about 0.05.

The particles of the alkaline earth metal carbonate, for example, calcium carbonate (e.g., PCC), may have a circularity (e.g. a high sensitivity (HS) circularity) of no less than about 0.8 (e.g. no less than about 0.80) and an elongation of no greater than about 0.2 (e.g. no greater than about 0.20), for example a circularity (e.g. a high sensitivity (HS) circularity) of no less than about 0.9 (e.g. no less than about 0.90) and an elongation of no greater than about 0.1 (e.g. no greater than about 0.10).

The (HS) circularity and the elongation may be determined, for example, using a Malvern morphogranulometer (supplied by Malvern instruments).

The (HS) circularity, C, is a measure of how close a shape is to a perfect circle and defines a relationship between the area of a particle equivalent circle and the real area of a particle:

$$C = \frac{4 π A}{{P}^{2}}$$

wherein A is the area of the particle and P is the perimeter of the particle. C takes values from 0 (irregular particle) to 1 (perfect circle).

The elongation, E, is a measure of the asymmetry of the particle shape:

$$E = 1 - \frac{W}{L}$$

wherein W is the particle width and L is the particle length. E takes values from 0 (a circle or perfect square) to 1 (a needle-like, long and fine particle).

The alkaline earth metal carbonate, for example, calcium carbonate (e.g., PCC), may be a spray-dried alkaline earth metal carbonate, for example, spray-dried calcium carbonate (e.g., spray-dried PCC). The alkaline earth metal carbonate, for example, calcium carbonate (e.g., PCC), may be spray-dried using a rotary disk or through a nozzle. Spray-drying the alkaline earth metal carbonate, for example, calcium carbonate (e.g., PCC), typically produces substantially spherical particles.

It may be that the particles of the alkaline earth metal carbonate, for example, calcium carbonate (e.g., PCC), are agglomerates (e.g. substantially spherical agglomerates), each agglomerate comprising a plurality of agglomerated primary alkaline earth metal carbonate crystallites (for example, agglomerated primary calcium carbonate (e.g., PCC) crystallites).

The alkaline earth metal carbonate, for example, calcium carbonate (e.g., PCC), may be porous. The term “porous” as used in relation to alkaline earth metal carbonates, such as calcium carbonate (and, in particular, PCC), refers to the presence of voids that allow gases and/or liquids to pass through the alkaline earth metal carbonate agglomerates or aggregates, for example, calcium carbonate (e.g., PCC). In particular, the term “porous” refers to the presence of voids in between the primary crystallites which are agglomerated to form alkaline earth metal (for example, calcium carbonate (e.g., PCC)) aggregates.

The alkaline earth metal carbonate, for example, calcium carbonate (e.g., PCC), may be surface-modified. For example, the alkaline earth metal carbonate, for example, calcium carbonate (e.g., PCC), may be coated. The coating may comprise (e.g. consist (e.g. essentially) of) a silane or salt thereof, for example, an organic silane. Additionally or alternatively, the coating may comprise (e.g. consist (e.g. essentially) of) a fatty acid or salt thereof. The fatty acid or salt thereof may contain 8 to 24 carbon atoms. For example, the coating may comprise (e.g. consist (e.g. essentially) of) caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, or salts thereof, or any mixture thereof. For example, the coating may comprise (e.g. consist (e.g. essentially) of) stearic acid, palmitic acid, a stearate, a palmate, or any mixture thereof. The level of coating may be from about 1 wt. % to about 20 wt. %, based on the total weight of the coated alkaline earth metal carbonate, for example, from about 1 wt. % to about 15 wt. %, or from about 1 wt. % to about 10 wt. %, or from about 1 wt. % to about 5 wt. %, or from about 2 wt. % to about 20 wt. %, or from about 2 wt. % to about 15 wt. %, or from about 2 wt. % to about 10 wt. %, or from about 2 wt. % to about 5 wt. %, or from about 3 wt. % to about 20 wt. %, or from about 3 wt. % to about 15 wt. %, or from about 3 wt. % to about 10 wt. %, or from about 3 wt. % to about 5 wt. %, or from about 3.5 wt. % to about 20 wt. %, or from about 3.5 wt. % to about 15 wt. %, or from about 3.5 wt. % to about 10 wt. %, or from about 3.5 wt. % to about 5 wt. %, or from about 4 wt. % to about 20 wt. %, or from about 4 wt. % to about 15 wt. %, or from about 4 wt. % to about 10 wt. %, or from about 4 wt. % to about 5 wt. %, or from about 5 wt. % to about 20 wt. %, or from about 5 wt. % to about 15 wt. %, or from about 5 wt. % to about 10 wt. %.

Coating the alkaline earth metal carbonate, for example, calcium carbonate (e.g., PCC), may improve the ease of application and/or the softness when a cosmetic composition comprising the alkaline earth metal carbonate, for example, calcium carbonate (e.g., PCC), is applied to the skin. Coating the alkaline earth metal carbonate, for example, calcium carbonate (e.g., PCC), with stearic acid, palmitic acid and/or salts thereof, for example, a fatty acid mixture comprising predominantly (e.g. consisting (e.g. essentially) of) stearic acid and palmitic acid, may particularly increase the ease of application and/or the softness when a cosmetic composition comprising the alkaline earth metal carbonate, for example, calcium carbonate (e.g., PCC), is applied to the skin. The alkaline earth metal carbonate, for example, calcium carbonate (e.g., PCC), may therefore be coated with from about 1 wt. % to about 20 wt. %, for example, from about 2 wt. % to about 10 wt. %, or from about 2 wt. % to about 6 wt. %, or from about 3 wt. % to about 6 wt. %, or from about 3 wt. % to about 5 wt. %, for example, about 4 wt. %, of stearic acid, palmitic acid and/or salts thereof, for example, a fatty acid mixture comprising predominantly (e.g. consisting (e.g. essentially) of) stearic acid and palmitic acid.

The term “coating” used herein is to be understood broadly, and is not limited, for example, to uniform coatings or to coatings which cover the entire surface area of a particle. Particles in which discrete regions of the surface are modified with a coating will be understood as being coated within the terms of certain embodiments of the present invention.

The alkaline earth metal carbonate, for example, calcium carbonate (e.g., PCC), may optionally further comprise one or more crystallization controller(s). The crystallization controller may, for example, be selected from the group consisting of polyacrylic acid, salts thereof and mixtures thereof, citric acid, sodium dioctylsulfosuccinate, polyaspartic acid and ethylenediaminetetraacetic acid (EDTA). The crystallization controller may, for example, be present in the alkaline earth metal carbonate, for example, calcium carbonate (e.g., PCC), in an amount no less than about 0.1 wt. % (by weight of alkaline earth metal carbonate). For example, the crystallization controller may be present in the alkaline earth metal carbonate, for example, calcium carbonate (e.g., PCC), in an amount no less than about 0.2 wt. %, or no less than about 0.25 wt. %, or no less than about 0.5 wt. %. For example, the crystallization controller may be present in the alkaline earth metal carbonate, for example, calcium carbonate (e.g., PCC), in an amount no greater than about 4 wt. %, or no greater than about 3 wt. %, or no greater than about 2.5 wt. %, or no greater than about 2 wt. %, or no greater than about 1 wt. %. Accordingly, the crystallization controller may be present in the alkaline earth metal carbonate, for example, calcium carbonate (e.g., PCC), in an amount from about 0.2 wt. % to about 4 wt. %, for example, from about 0.25 wt. % to about 4 wt. %, or from about 0.5 wt. % to about 4 wt. %, or from about 0.2 wt. % to about 3 wt. %, or from about 0.25 wt. % to about 3 wt. %, or from about 0.5 wt. % to about 3 wt. %, or from about 0.2 wt. % to about 2.5 wt. %, or from about 0.25 wt. % to about 2.5 wt. %, or from about 0.5 wt. % to about 2.5 wt. %, or from about 0.2 wt. % to about 2 wt. %, or from about 0.25 wt. % to about 2 wt. %, or from about 0.5 wt. % to about 2 wt. %.

When polyacrylic acid, salts thereof and mixtures thereof are present in the alkaline earth metal carbonate, for example, calcium carbonate (e.g., PCC), particles, generally the molecular weight of the polyacrylic acid or its salt, in particular sodium salt, is from about 500 and up to about 15,000 g/mol. The molecular weight may, for example, be equal to or larger than about 500 g/mol or equal to or larger than about 700 g/mol or equal to or larger than about 1000 g/mol. Generally, the molecular weight is equal to or less than about 15,000 g/mol, or equal to or less than about 10,000 g/mol or equal to or less than about 5000 g/mol. For example, the molecular weights may be from about 1000 to about 3500 g/mol. If the polyacrylic acid is present as salt, such as the sodium salt, the degree of acid neutralization by its cation, in particular sodium, can be from 0 to 100%. For instance, around 70% of the acid groups may be neutralized. The crystallization controlled may thus have a pH ranging from about 5 to about 6. For instance, around 100% of the acid groups may be neutralized and the crystallization controller may have a pH ranging from about 6.5 to about 10.

### Cosmetic Composition

The cosmetic composition typically comprises the alkaline earth metal carbonate, for example, calcium carbonate (e.g., PCC), and at least one other component.

The cosmetic composition may comprise no less than about 0.5 wt. %, for example, no less than about 1 wt. %, or no less than about 2 wt. %, or no less than about 5 wt. %, or no less than about 10 wt. %, or no less than about 20 wt. %, of the alkaline earth metal carbonate, for example, calcium carbonate (e.g., PCC). The cosmetic composition may comprise no greater than about 90 wt. %, for example, no greater than about 80 wt. %, or no greater than about 70 wt. %, or no greater than about 60 wt. %, or no greater than about 50 wt. %, or no greater than about 40 wt. %, or no greater than about 30 wt. %, or no greater than about 20 wt. %, or no greater than about 10 wt. %, or no greater than about 5 wt. %, of the alkaline earth metal carbonate, for example, calcium carbonate (e.g., PCC). The cosmetic composition may comprise from about 0.5 wt. % to about 90 wt. %, for example, from about 1 wt. % to about 90 wt. %, or from about 2 wt. % to about 90 wt. %, or from about 5 wt. % to about 90 wt. %, or from about 10 wt. % to about 90 wt. %, or from about 20 wt. % to about 90 wt. %, or from about 0.5 wt. % to about 80 wt. %, or from about 1 wt. % to about 80 wt. %, or from about 2 wt. % to about 80 wt. %, or from about 5 wt. % to about 80 wt. %, or from about 10 wt. % to about 80 wt. %, or from about 20 wt. % to about 80 wt. %, or from about 0.5 wt. % to about 70 wt. %, or from about 1 wt. % to about 70 wt. %, or from about 2 wt. % to about 70 wt. %, or from about 5 wt. % to about 70 wt. %, or from about 10 wt. % to about 70 wt. %, or from about 20 wt. % to about 70 wt. %, or from about 0.5 wt. % to about 60 wt. %, or from about 1 wt. % to about 60 wt. %, or from about 2 wt. % to about 60 wt. %, or from about 5 wt. % to about 60 wt. %, or from about 10 wt. % to about 60 wt. %, or from about 20 wt. % to about 60 wt. %, or from about 0.5 wt. % to about 50 wt. %, or from about 1 wt. % to about 50 wt. %, or from about 2 wt. % to about 50 wt. %, or from about 5 wt. % to about 50 wt. %, or from about 10 wt. % to about 50 wt. %, or from about 20 wt. % to about 50 wt. %, or from about 0.5 wt. % to about 40 wt. %, or from about 1 wt. % to about 40 wt. %, or from about 2 wt. % to about 40 wt. %, or from about 5 wt. % to about 40 wt. %, or from about 10 wt. % to about 40 wt. %, or from about 20 wt. % to about 40 wt. %, or from about 0.5 wt. % to about 30 wt. %, or from about 1 wt. % to about 30 wt. %, or from about 2 wt. % to about 30 wt. %, or from about 5 wt. % to about 30 wt. %, or from about 10 wt. % to about 30 wt. %, or from about 20 wt. % to about 30 wt. %, or from about 0.5 wt. % to about 20 wt. %, or from about 1 wt. % to about 20 wt. %, or from about 2 wt. % to about 20 wt. %, or from about 5 wt. % to about 20 wt. %, or from about 10 wt. % to about 20 wt. %, or from about 0.5 wt. % to about 10 wt. %, or from about 1 wt. % to about 10 wt. %, or from about 2 wt. % to about 10 wt. %, or from about 5 wt. % to about 10 wt. %, or from about 0.5 wt. % to about 5 wt. %, or from about 1 wt. % to about 5 wt. %, or from about 2 wt. % to about 5 wt. %, of the alkaline earth metal carbonate, for example, calcium carbonate (e.g., PCC).

The at least one other component may be selected from: water or an aqueous solution; a polymer; an oil, such as an oil derived from a plant source, an animal source or a mineral (e.g. petrochemical) source; a wax, such as a wax derived from a plant source, an animal source or a mineral (e.g. petrochemical) source; a fatty acid or salt thereof; an amino acid or a polypeptide such as a protein; a sugar or carbohydrate; a mineral other than the alkaline earth metal carbonate, for example, an iron oxide, zinc oxide, talc, mica, wollastonite, diatomaceous earth or a clay mineral (e.g. kaolinite, montmorillonite or illite); a pigment; a perfume. The cosmetic composition may contain two or more (i.e. different components) other than the alkaline earth metal carbonate.

The cosmetic composition may be an anhydrous cosmetic composition, i.e. a cosmetic composition which is substantially (e.g. entirely) water-free. For example, the cosmetic composition may comprise no greater than about 1 wt. %, for example, no greater than about 0.1 wt. %, or no greater than about 0.01 wt. %, water. Alternatively, the cosmetic composition may be a non-anhydrous (i.e. hydrous or water-containing) cosmetic composition. For example, the cosmetic composition may comprise no less than about 1 wt. %, for example, greater than about 1 wt. %, or greater than about 5 wt. %, water.

The cosmetic composition may be a water-based cosmetic composition. Alternatively, the cosmetic composition may be an oil-based cosmetic composition. Further alternatively, the cosmetic composition may contain both water and an oil, for example, in the form of an emulsion.

The cosmetic composition may be a dry cosmetic composition. For example, the cosmetic composition may be a powder, such as a loose powder or a compressed powder.

The cosmetic composition may be a wet cosmetic composition. For example, the cosmetic composition may be a cream, gel, gel-cream, emulsion, lotion, fluid, milk or serum.

The cosmetic composition may be a solid cosmetic composition. For example, the cosmetic composition may be a solid stick.

The cosmetic composition may be a liquid cosmetic composition. For example, the cosmetic composition may be a cream, lotion, fluid, milk or serum.

The cosmetic composition may be semi-solid cosmetic composition. For example, the cosmetic composition may be a gel.

The cosmetic composition may comprise a mixture of one or more liquid components and one or more solid components. For example, the cosmetic composition may be a paste or slurry.

The cosmetic composition is typically for topical application to the skin. For example, the cosmetic composition may be for topical application to the skin of the head and neck, for example, facial skin (which may include the lips). Additionally or alternatively, the cosmetic composition may be for topical application to the skin of the body (for example, of the limbs, such as the arms or legs, and/or the décolletage).

The cosmetic composition may be, or may be suitable for use in, a form of make-up. For example, the cosmetic composition may be or may be suitable for use in: a primer; a concealer; a foundation; a rouge or a blusher; a bronzer; a highlighter; an eyeshadow; an eyebrow pencil, cream, wax, gel or powder; an eyeliner pencil, gel or liquid; a mascara; a lipstick, lip gloss, lip balm or lip liner; or a face powder, setting powder or setting spray.

Alternatively, the cosmetic composition may be, or may be suitable for use in, a form of skincare. For example, the cosmetic composition may be or may be suitable for use in: a moisturiser; a serum; a toner; a skin oil (for example, a facial oil); or a sunscreen.

### Soft Focus Effect

It has been found that including the alkaline earth metal carbonate in the cosmetic composition can improve the soft focus effect of the cosmetic composition, i.e. the soft focus effect achieved when the cosmetic composition is applied to the skin.

The soft focus effect, when the cosmetic composition is applied to the skin, may reduce the appearance of (i.e. render less visible or noticeable) skin imperfections. For example, the soft focus effect may reduce the appearance of (i.e. render less visible or noticeable) wrinkles, otherwise known as rhytids, i.e. folds, ridges or creases in the skin. Additionally or alternatively, the soft focus effect may reduce the appearance of (i.e. render less visible or noticeable) blemishes such as liver spots (also known as age spots), dark circles, freckles, pimples, blackheads, whiteheads, enlarged pores, melasma, scars, hyperpigmentation and/or bruises.

The soft focus effect of the alkaline earth metal carbonate may be evaluated by trained panellists. For example, the alkaline earth metal carbonate may be incorporated into an oil phase matrix, having a refractive index which substantially matches that of the alkaline earth metal carbonate (e.g. 1.5 for calcium carbonate). Dicaprylyl carbonate is a suitable oil for testing calcium carbonate. A mixture of 50%-50% by weight alkaline earth metal carbonate to oil may be used. A measured amount of the mixture may be applied and spread over a defined area of the panellists' skin, for example in the hollow of the wrist where fine lines are visible. The panellists can rate the soft focus effect, for example on a scale of 0 (corresponding to the soft focus effect achieved using oil alone) to 5 (corresponding to the soft focus effect achieved using a reference synthetic soft focus agent such as silica SB700 (available from Myoshi Kasei, Inc., Japan)).

It will be understood that the invention is not limited to the embodiments described above and various modifications and improvements can be made without departing from the concepts described herein. Except where mutually exclusive, any of the features may be employed separately or in combination with any other features and the disclosure extends to and includes all combinations and sub-combinations of one or more features described herein.

For the avoidance of doubt, the present application is directed to the subject-matter described in the following numbered paragraphs:

1. Alkaline earth metal carbonate for use in a cosmetic composition, the alkaline earth metal carbonate having a BET specific surface area of at least about 60 m²/g, wherein particles of the alkaline earth metal carbonate are substantially spherical.

2. The alkaline earth metal carbonate according to paragraph 1, wherein the alkaline earth metal carbonate is calcium carbonate, for example, precipitated calcium carbonate.

3. The alkaline earth metal carbonate according to paragraph 1 or paragraph 2, wherein the alkaline earth metal carbonate:
(a) has a BET specific surface area from about 60 m²/g to about 200 m²/g, for example, from about 64 m²/g to about 140 m²/g;
(b) has a D₅₀@T₉₀ of at least about 6 μm, for example, from about 6 μm to about 20 μm; and/or
(c) has an agglomeration rate of at least about 85%, for example, at least about 90%, or at least about 92%, the agglomeration rate A being defined by

$$A = \frac{{D}_{50} @ {T}_{90}}{{D}_{50} @ {T}_{45}} \times 100 \% .$$

4. The alkaline earth metal carbonate according to any preceding paragraph, wherein the particles of the alkaline earth metal carbonate have an HS circularity of at least about 0.8 and/or an elongation of no greater than about 0.2.

5. The alkaline earth metal carbonate according to any preceding paragraph, wherein the particles of the alkaline earth metal carbonate are agglomerates each comprising a plurality of agglomerated primary alkaline earth metal carbonate crystallites.

6. The alkaline earth metal carbonate according to any preceding paragraph, wherein the alkaline earth metal carbonate is coated, for example, with a coating comprising a fatty acid or salt thereof, such as stearic acid, palmitic acid, a stearate or a palmate, or any mixture thereof.

7. The alkaline earth metal carbonate according to paragraph 6, wherein the alkaline earth metal carbonate comprises from about 1 wt. % to about 20 wt. %, for example, from about 2 wt. % to about 15 wt. %, or from about 3.5 wt. % to about 15 wt. %, of the fatty acid or salt thereof, such as the stearic acid, palmitic acid, the stearate or the palmate, or any mixture thereof.

8. A cosmetic composition comprising the alkaline earth metal carbonate according to any preceding paragraph and at least one other component.

9. Use of an alkaline earth metal carbonate having a BET specific surface area of at least about 60 m²/g in a cosmetic composition to improve the soft focus effect of the cosmetic composition, wherein particles of the alkaline earth metal carbonate are substantially spherical.

10. The use according to paragraph 9, wherein the alkaline earth metal carbonate:
(a) is calcium carbonate, for example, precipitated calcium carbonate;
(b) has a BET specific surface area from about 60 m²/g to about 200 m²/g, for example, from about 64 m²/g to about 140 m²/g;
(c) has a D₅₀@T₉₀ of at least about 6 μm, for example, from about 6 μm to about 20 μm; and/or
(d) has an agglomeration rate of at least about 85%, for example, at least about 90%, or at least about 92%, the agglomeration rate A being defined by

$$A = \frac{{D}_{50} @ {T}_{90}}{{D}_{50} @ {T}_{45}} \times 100 \% .$$

11. The use according to paragraph 9 or paragraph 10, wherein the particles of the alkaline earth metal carbonate have an HS circularity of at least about 0.8 and/or an elongation of no greater than about 0.2.

12. The use according to any of paragraphs 9 to 11, wherein the particles of the alkaline earth metal carbonate are agglomerates each comprising a plurality of agglomerated primary alkaline earth metal carbonate crystallites.

13. The use according to any of paragraphs 9 to 12, wherein the alkaline earth metal carbonate is coated, for example, with a coating comprising a fatty acid or salt thereof, such as stearic acid, palmitic acid, a stearate, or a palmate, or any mixture thereof.

14. The use according to paragraph 13, wherein the alkaline earth metal carbonate comprises from about 1 wt. % to about 10 wt. %, for example, from about 2 wt. % to about 5 wt. %, or from about 3.5 wt. % to about 4.5 wt. %, of the fatty acid or salt thereof, such as the stearic acid, palmitic acid, stearate, or palmate, or any mixture thereof.

15. A method for reducing the appearance of skin imperfections, such as wrinkles, the method comprising applying (a) the alkaline earth metal carbonate according to any of paragraphs 1 to 7, or (b) the cosmetic composition according to paragraph 8, to the skin.

### EXAMPLES

### Example 1

Twenty-one different example precipitated calcium carbonates (PCC1 to PCC21), as set out in Table 1, were obtained or prepared.

PCC1, PCC2 and PCC9 to PCC21 were prepared by the following method. A milk of lime consisting of calcium hydroxide (Ca(OH)₂) at a concentration of 80 g/L was placed in a 350 L carbonator. Citric acid was added at a concentration of 2 wt. % vs. PCC. Carbonation reaction was performed at 15° C. The CO₂ flow rate was 90 Nm³/h. When the reaction was completed and the pH reached 7, an overcarbonation was performed at 26 Nm³/h to ensure that all of the Ca(OH)₂ had been carbonated. The resultant product was optionally functionalized with a stearin (a mixture of fatty acids, mostly palmitic and stearic acids) as set out in Table 1. If functionalised with the stearin, the fatty acid was added in emulsion with an alkali such as NH₄OH or NaOH. The product was sieved to remove the bigger parts. Finally, the product was spray-dried using either a rotary disk method or through a nozzle, as set out in Table 1. For the spray-drying, the inlet temperature was 300° C., while the outlet temperature was 105° C.

PCC3 to PCC8 were different commercially available precipitated calcium carbonates also prepared by spray-drying. PCC2, PCC4, PCC6 and PCC8 were coated with maltodextrin.

TABLE 1

| PCC | Coating | Drying method | Particle shape |
|---|---|---|---|
| PCC1 | None | Spray dried | Spherical |
| | | (disk) | |
| PCC2 | maltodextrin | Spray dried | Spherical |
| | | (disk) | |
| PCC3 | None | Spray dried | Ellipsoid |
| | | (disk) | |
| PCC4 | 2 wt. % | Spray dried | Ellipsoid |
| | maltodextrin | (disk) | |
| PCC5 | None | Spray dried | Ellipsoid |
| | | (disk) | |
| PCC6 | 2 wt. % | Spray dried | Ellipsoid |
| | maltodextrin | (disk) | |
| PCC7 | None | Spray dried | Ellipsoid |
| | | (disk) | |
| PCC8 | 2 wt. % | Spray dried | Ellipsoid |
| | maltodextrin | (disk) | |
| PCC9 | None | Spray dried | Spherical |
| | | (disk) | |
| PCC10 | 2 wt. % | Spray dried | Spherical |
| | stearin | (disk) | |
| PCC11 | None | Spray dried | Spherical |
| | | (nozzle) | |
| PCC12 | 2 wt. % | Spray dried | Spherical |
| | stearin | (nozzle) | |
| PCC13 | 4 wt. % | Spray dried | Spherical |
| | stearin | (nozzle) | |
| PCC14 | 4 wt. % | Spray dried | Spherical |
| | stearin | (nozzle) | |
| PCC15 | None | Spray dried | Spherical |
| | | (nozzle) | |
| PCC16 | None | Spray dried | Spherical |
| | | (nozzle) | |
| PCC17 | None | Spray dried | Spherical |
| | | (nozzle) | |
| PCC18 | None | Spray dried | Spherical |
| | | (nozzle) | |
| PCC19 | 4 wt. % | Spray dried | Spherical |
| | stearin | (disk) | |
| PCC20 | 4 wt. % | Spray dried | Spherical |
| | stearin | (nozzle) | |
| PCC21 | None | Spray dried | Spherical |
| | | (disk) | |

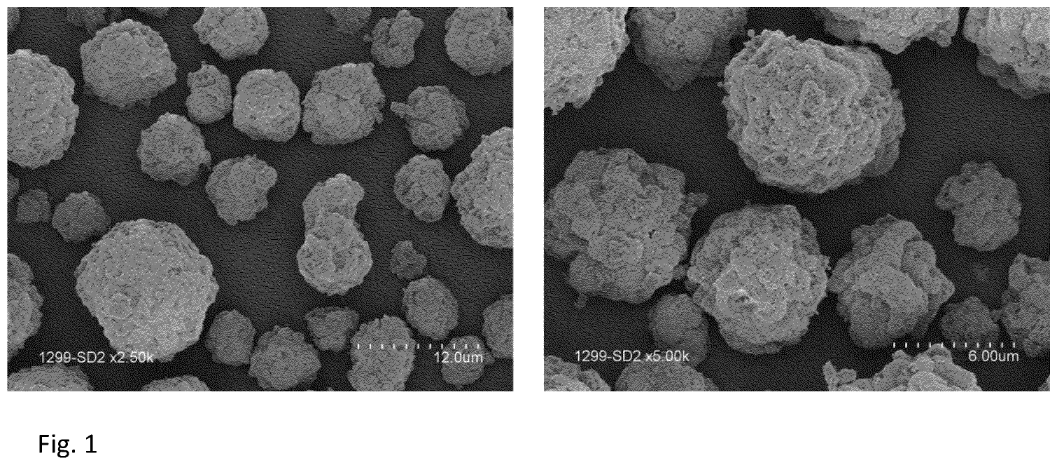

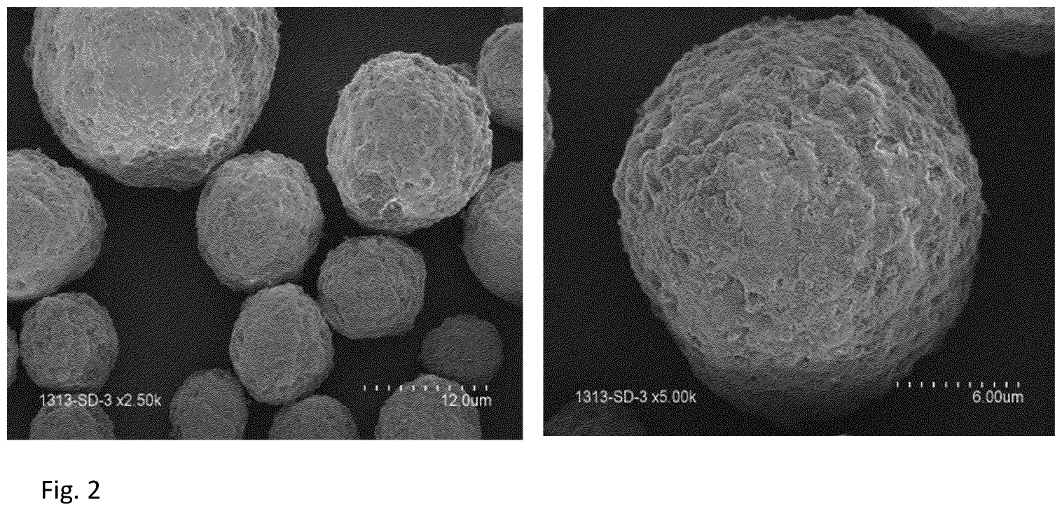

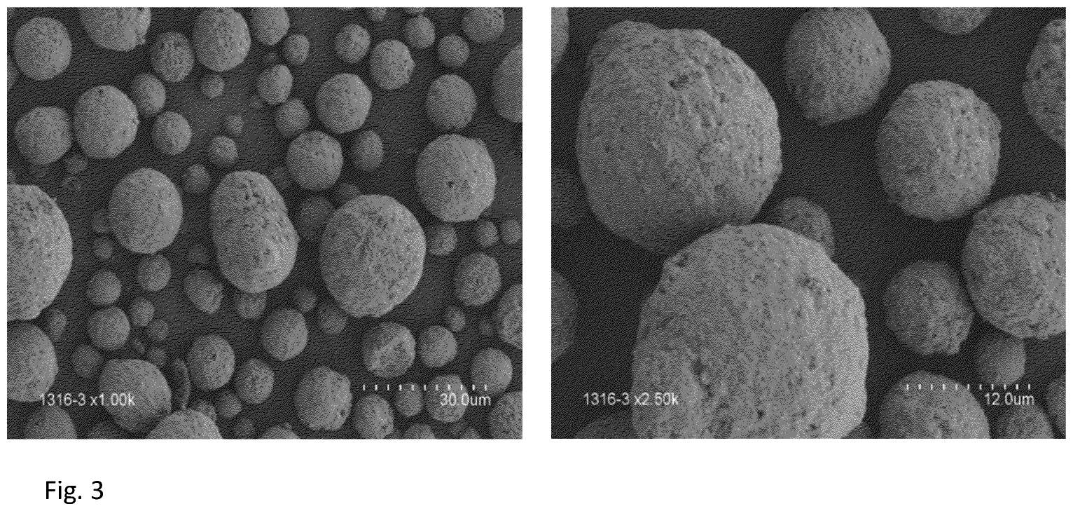

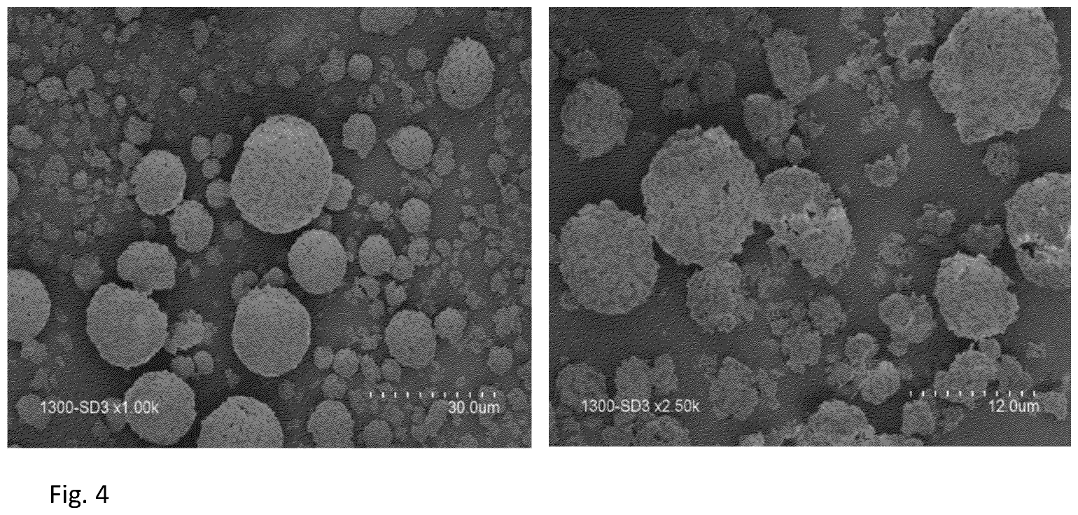

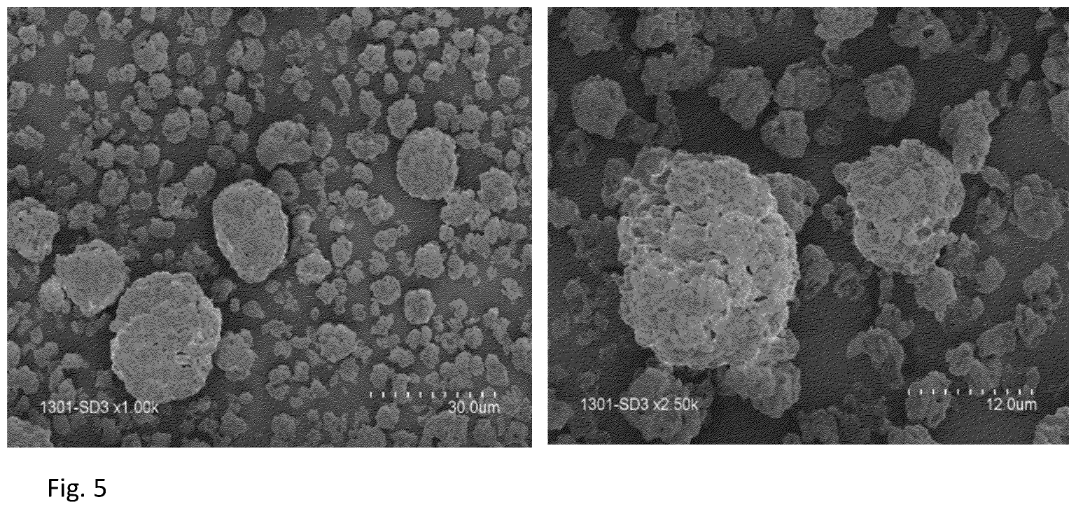

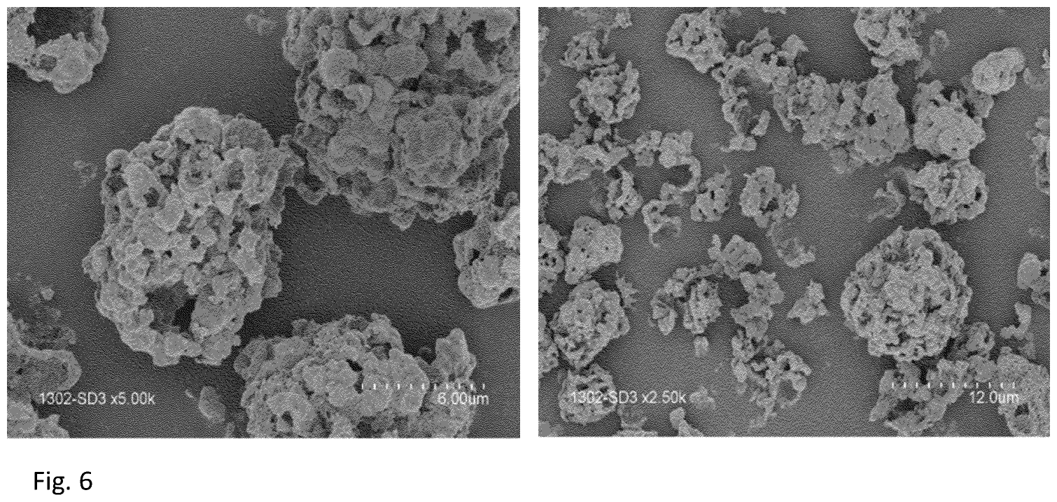

Samples of PCC1, PCC4, PCC6, PCC7, PCC9 and PCC13 were observed in a Scanning Electron Microscope (Hitachi S4800 Field Emission Scanning Electron Microscope with a secondary electron and backscattered electron detector, an accelerating voltage of 0.1 kV to 30 kV, maximum magnification of 800 000×, resolution of 1 nm at 15 kV, and a transmission option for the observation of fine samples at 30 kV) following sputtering with platinum (Pt). FIG. 1 shows SEM images obtained for PCC1. FIG. 2 shows SEM images obtained for PCC9. FIG. 3 shows SEM images obtained for PCC13. FIG. 4 shows SEM images obtained for PCC4. FIG. 5 shows SEM images obtained for PCC6. FIG. 6 shows SEM images obtained for PCC7.

The (HS) circularity and the elongation of the samples PCC1 to PCC21 were determined, using a Malvern morphogranulometer (supplied by Malvern instruments). Samples PCC1, PCC2, and PCC9 to PCC21 have a circularity of more than 0.80 and an elongation of less than 0.20 (for example, PCC13 has a circularity of 0.92 and an elongation of 0.05). Samples PCC3 to PCC8 have a circularity of less than 0.80 and/or an elongation of more than 0.20 (for example, PCC 7 has a circularity of 0.78 and an elongation of 0.21).

For each of PCC1 to PCC21, BET surface area, D₅₀@T₉₀ and agglomeration rate were measured.

The BET surface area was measured using the BET method, ISO 9277.

D₅₀@T₄₅, D₅₀@T₉₀ and the agglomeration rate were determined by the “dispersion evaluation by laser method” (DELM) using laser granulometry using the Malvern Mastersizer 2000 (as supplied by Malvern instruments). The measurement cell of the Malvern Mastersizer 2000 was filled, in turn, with a mixture of isopropanol and a small amount of each PCC. The obscuration was then measured and the mixture adjusted so that the obscuration was between 1 and 3% (for the measurement to be reliable). The mixture was then subjected to mechanical stirring at about 1500 rpm for about 4 minutes and 30 seconds. The particle size distribution (PSD) was measured 45 times during this time. The 45^{th} PSD, or the nearest representative PSD, was recorded. A PSD was considered as representative when it did not comprise agglomerates above 500 μm which could be attributed to air bubbles. The D₅₀ value obtained from the recorded PSD after stirring the mixture was recorded as the D₅₀@T₄₅.

Following measurement of D₅₀@T₄₅, the stirred mixture was subjected to pulsed ultrasound for about 4 minutes and 30 seconds at a total intensity of about 1000 J to 1500 J. The pulsed ultrasound was applied using the Vibracell 75186 sonicator (available from Thermo Fisher Scientific, Inc., USA), operated at a power of 130 W and a frequency of 20 kHz, with a probe having a 3 mm stepped tip. The PSD was again measured 45 times during this time. The 45^{th} PSD, or the nearest representative PSD, was recorded. A PSD was considered as representative when it did not comprise agglomerates above 500 μm which could be attributed to air bubbles. The D₅₀ value obtained from the recorded PSD after application of the pulsed ultrasound was recorded as the D₅₀@T₉₀. The intensity of the pulsed ultrasound was selected by measuring a PCC reference sample and ensuring that the D₅₀@T₉₀ was +/−10% of what had been obtained previously.

The agglomeration rate, A, was calculated according to:

$$A = \frac{{D}_{50} @ {T}_{90}}{{D}_{50} @ {T}_{45}} \times 100 \% .$$

The results are shown in Table 2.

TABLE 2

| | BET surface | D₅₀ @ | Agglomeration |
|---|---|---|---|
| PCC | area (m²/g) | T₉₀ (μm) | rate (%) |
| PCC1 | 106 | 9.8 | 97.8 |
| PCC2 | 98 | 8.6 | 97.5 |
| PCC3 | 20 | 1.8 | 24.7 |
| PCC4 | 17 | 6.2 | 56.0 |
| PCC5 | 57 | 4.7 | 55.3 |
| PCC6 | 51 | 5.2 | 57.7 |
| PCC7 | 10 | 4.5 | 84.4 |
| PCC8 | 7 | 6.0 | 69.9 |
| PCC9 | 134 | 18.3 | 99.8 |
| PCC10 | 64 | 18.0 | 97.6 |
| PCC11 | 80 | 15.7 | 100.4 |
| PCC12 | 78 | 16.0 | 99.3 |
| PCC13 | 76 | 15.2 | 99.7 |
| PCC14 | 78 | 16.1 | 99.3 |
| PCC15 | 76 | 9.2 | 98.5 |
| PCC16 | 77 | 9.7 | 94.1 |
| PCC17 | 80 | 13.2 | 97.9 |
| PCC18 | 80 | 8.6 | 93.7 |
| PCC19 | 69 | 8.0 | 99.4 |
| PCC20 | 68 | 15.2 | 98.6 |
| PCC21 | 82 | 14.0 | 99.7 |

The soft focus effect and ease of application of each of PCC1 to PCC21 was evaluated by a panel of 5 trained panellists by the following method. A 50%-50% mixture of each PCC with dicaprylyl carbonate oil was made. A measured amount of the mixture was applied and spread over a defined area of the panellists' skin in the hollow of the wrist where fine lines were visible. The panellists rated the soft focus effect (i.e. the ability to reduce the appearance of the fine lines) on a scale of 0 (corresponding to the soft focus effect achieved using the oil alone) to 5 (corresponding to the soft focus effect achieved using a reference synthetic soft focus agent, silica SB700 (available from Myoshi Kasei, 10 Inc., Japan)). The panellists also rated the ease of application on a scale of 0 (corresponding to least easy to apply) to 5 (corresponding to easiest to apply). The results are presented in Table 3.

TABLE 3

| | Soft focus | Ease of | Soft |
|---|---|---|---|
| | effect | application | focus |
| PCC | (scale 0 to 5) | (scale of 0 to 5) | performance |
| PCC1 | 5 | 2 | Acceptable |
| PCC2 | 5 | 3 | Acceptable |
| PCC3 | 1 | 1 | Not acceptable |
| PCC4 | 1 | 1 | Not acceptable |
| PCC5 | 3 | 3 | Not acceptable |
| PCC6 | 3 | 3 | Not acceptable |
| PCC7 | 1 | 1 | Not acceptable |
| PCC8 | 1 | 1 | Not acceptable |
| PCC9 | 5 | 2 | Acceptable |
| PCC10 | 5 | 5 | Acceptable |
| PCC11 | 5 | 3 | Acceptable |
| PCC12 | 5 | 3 | Acceptable |
| PCC13 | 5 | 5 | Acceptable |
| PCC14 | 5 | 5 | Acceptable |
| PCC15 | 5 | 0 | Acceptable |
| PCC16 | 5 | 1 | Acceptable |
| PCC17 | 5 | 0 | Acceptable |
| PCC18 | 5 | 2 | Acceptable |
| PCC19 | 5 | 5 | Acceptable |
| PCC20 | 5 | 5 | Acceptable |
| PCC21 | 5 | 3 | Acceptable |

Based on the results shown in Table 3, PCC1, PCC2 and PCC9 to PCC21, all of which achieved a score of 5 for the soft focus effect, are considered have an acceptable soft focus effect. PCC3 to PCC8, all of which achieved a score of 3 or lower for the soft focus effect, are considered to have an unacceptable soft focus effect.

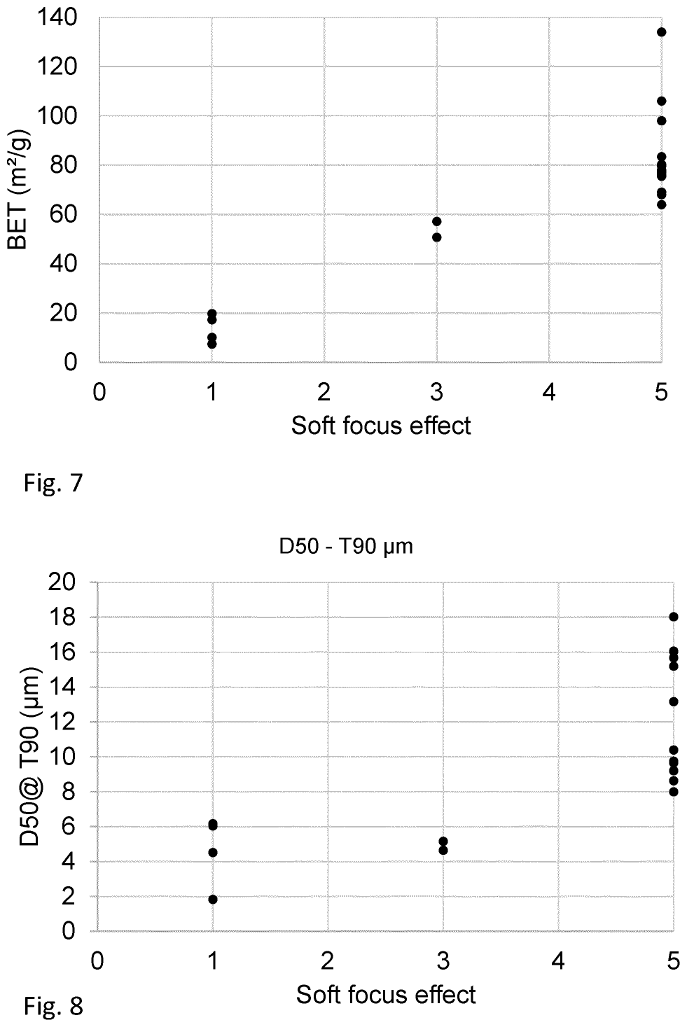

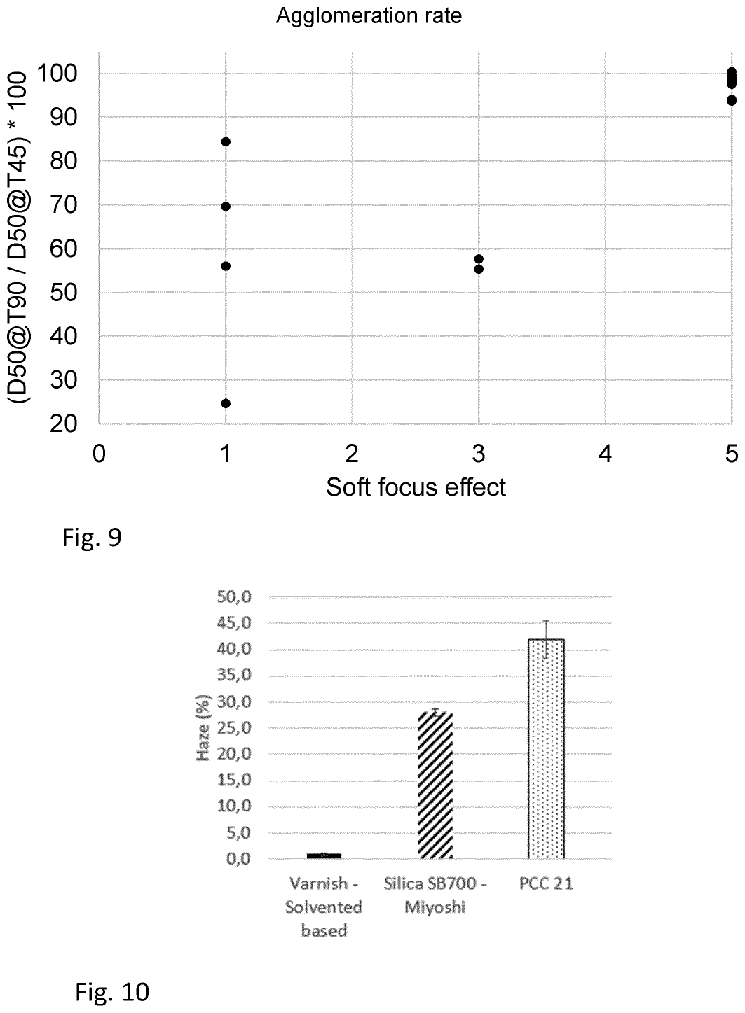

FIGS. 7, 8 and 9 show plots of the BET surface area, D₅₀@T₉₀ and agglomeration rate as a function of the soft focus effect. From these plots, it can be seen that an improved soft focus effect is correlated with higher (e.g. no less than about 60 m²/g) BET surface areas, higher (e.g. no less than about 6 μm, or no less than about 8 μm) values of D₅₀@T₉₀, and higher (e.g. no less than about 85%, for example, no less than about 90%) agglomeration rates.

By comparing the results of Tables 1 and 3, it can also be seen that an improved ease of application is correlated with use of a stearin coating, and that the best ease of application is achieved when a 4 wt. % stearin coating is used.

### Example 2

In addition to the panellists' test, the improved soft focus effect of the invention has further been shown using a “Haze” spectrophotometer measurement, as described below.

### 1. Sample Preparation

For the spectrophotometer measurement, particles of a sample need to be incorporated in a resin capable of forming a hard film on a support.

Here is described the basic formulation used, capable of forming a hard film when drying. It was made with a nitrocellulose lacquer that forms a smooth and transparent hard film. A smooth-surfaced and transparent film is necessary because any coloration or roughness of the film will affect the results. The hard film also safeguards against accidental damage to the film surface by burnishing or contamination of foreign objects.

The mixture is prepared using the formulation shown in Table 4, with the soft focus sample particles being incorporated at 2.5% weight. The reference for the study is 100% nail varnish.

TABLE 4

| Component | Description | Supplier | wt. % |
|---|---|---|---|
| Nail | Transparent | International | 97.5 |
| varnish | nitrocellulose | Lacquers Group | |
| | lacquer | SA, Luxembourg | |
| Soft focus material | White powder | — | 2.5 |

The formulations are prepared using a SpeedMixer DAC 150.1 FV (operated for 2 minutes at 2000 rpm, repeated twice) to ensure that a good dispersion of soft focus material is achieved and also that the preparation is homogenous.

### 2. Film Application

A film of 50 μm thickness is applied onto a transparent polyester film substrate (byko-charts from BYK, i.e. BYK-Gardner GmbH, Germany) using an automatic applicator (speed: 10 mm/s; difference: 0; move: 170 mm) with glass plate (byko-drive XL from BYK) and a 50 μm-thick drawbar from BYK.

The sheet is then dried for 24 hours minimum in a conditioned room (at a temperature of 23° C., and ˜ 50% humidity).

### 3. Spectrophotometer Measurements

The Haze of the film is measured using a PerkinElmer Lambda 650S spectrophotometer (available from PerkinElmer Inc., USA) with a 60 mm integration sphere (method: Haze ASTM D-1003-00). The Haze measurement describes the amount of light scattered when light passes through the sample, which corresponds to the ratio of the diffuse transmission to the total transmission.

The higher the haze measurement value, the higher the soft focus effect.

For each material, three different samples were measured to generate an average value with standard deviation.

PCC21 and the reference synthetic soft focus agent, silica SB700 (available from Myoshi Kasei, Inc., Japan), were analysed accordingly. The results are shown in FIG. 10.

1. An alkaline earth metal carbonate for use in a cosmetic composition, the alkaline earth metal carbonate having a BET specific surface area of at least about 60 m²/g, wherein particles of the alkaline earth metal carbonate are substantially spherical.

2. The alkaline earth metal carbonate according to claim 1, wherein the alkaline earth metal carbonate is calcium carbonate.

3. The alkaline earth metal carbonate according to claim 1, wherein the alkaline earth metal carbonate:
(a) has a BET specific surface area from about 60 m²/g to about 200 m²/g, for example, from about 64 m²/g to about 140 m²/g;
(b) has a D₅₀@T₉₀ of at least about 6 μm, for example, from about 6 μm to about 20 μm; or
(c) has an agglomeration rate of at least about 85%, for example, at least about 90%, or at least about 92%, the agglomeration rate A being defined by
$$A = \frac{{D}_{50} @ {T}_{90}}{{D}_{50} @ {T}_{45}} \times 100 \% .$$

4. The alkaline earth metal carbonate according to claim 1, wherein the particles of the alkaline earth metal carbonate have an HS circularity of at least about 0.8 and an elongation of no greater than about 0.2.

5. The alkaline earth metal carbonate according to claim 1, wherein the particles of the alkaline earth metal carbonate are agglomerates each comprising a plurality of agglomerated primary alkaline earth metal carbonate crystallites.

6. The alkaline earth metal carbonate according to claim 1, wherein the alkaline earth metal carbonate is coated with a coating comprising a fatty acid or salt thereof, such as stearic acid, palmitic acid, a stearate or a palmate, or any mixture thereof.

7. The alkaline earth metal carbonate according to claim 6, wherein the alkaline earth metal carbonate comprises from about 1 wt. % to about 20 wt. % of the fatty acid or salt thereof.

8. A cosmetic composition comprising the alkaline earth metal carbonate according to claim 1 and at least one other component.

9. A cosmetic composition comprising, an alkaline earth metal carbonate having a BET specific surface area of at least about 60 m²/g wherein particles of the alkaline earth metal carbonate are substantially spherical.

10. The cosmetic composition according to claim 9, wherein the alkaline earth metal carbonate:
(a) is calcium carbonate;
(b) has a BET specific surface area from about 60 m²/g to about 200 m²/g;
(c) has a D₅₀@T₉₀ of at least about 6 μm; or
(d) has an agglomeration rate of at least about 85, the agglomeration rate A being defined by
$$A = \frac{{D}_{50} @ {T}_{90}}{{D}_{50} @ {T}_{45}} \times 100 \% .$$

11. The cosmetic composition according to claim 9, wherein the particles of the alkaline earth metal carbonate have an HS circularity of at least about 0.8 and an elongation of no greater than about 0.2.

12. The cosmetic composition according to claim 9, wherein the particles of the alkaline earth metal carbonate are agglomerates each comprising a plurality of agglomerated primary alkaline earth metal carbonate crystallites.

13. The cosmetic composition according to claim 9, wherein the alkaline earth metal carbonate is coated with a coating comprising a fatty acid or salt thereof, such as stearic acid, palmitic acid, a stearate, or a palmate, or any mixture thereof.

14. The cosmetic composition according to claim 13, wherein the alkaline earth metal carbonate comprises from about 1 wt. % to about 10 wt. %, for example of the fatty acid or salt thereof.

15. A method for reducing the appearance of wrinkles or other skin imperfections, the method comprising applying (a) the alkaline earth metal carbonate according to claim 1 to the skin.

16. The alkaline earth metal carbonate according to claim 1, wherein the alkaline earth metal carbonate is precipitated calcium carbonate having a BET specific surface area from about 64 m²/g to about 140 m²/g.

17. The alkaline earth metal carbonate according to claim 16, wherein the alkaline earth metal carbonate has a D₅₀@T₉₀ from about 6 μm to about 20 μm.

18. The alkaline earth metal carbonate according to claim 17, wherein the alkaline earth metal carbonate has a D₅₀@T₉₀ from about 6 μm to about 20 μm.

19. The alkaline earth metal carbonate according to claim 17, wherein
the particles of the alkaline earth metal carbonate are agglomerates each comprising a plurality of agglomerated primary alkaline earth metal carbonate crystallites; and
the alkaline earth metal carbonate has an agglomeration rate of at least about 92%, the agglomeration rate A being defined by
$$A = \frac{{D}_{50} @ {T}_{90}}{{D}_{50} @ {T}_{45}} \times 100 \% .$$

20. The alkaline earth metal carbonate according to claim 19 wherein the alkaline earth metal carbonate is coated with a coating comprising a fatty acid or salt thereof, such as stearic acid, palmitic acid, a stearate or a palmate, or any mixture thereof; and
wherein the alkaline earth metal carbonate comprises from about 3.5 wt. % to about 15 wt. %, of the fatty acid or salt thereof.
